(19) United States
(12) Patent Application Publication
(10) Pub. No.: US 2015/0013381 A1
(43) Pub. Date: Jan. 15, 2015

(54) DISINFECTING JEWELRY HOLDER AND METHOD

(71) Applicant: Shannon K. Duffy, Chandler, AZ (US)

(72) Inventor: Shannon K. Duffy, Chandler, AZ (US)

(21) Appl. No.: 13/941,995

(22) Filed: Jul. 15, 2013

(51) Int. Cl. A44C 7/00; A61L 2/18

(52) U.S. Cl. CPC A44C 7/003; A61L 2/18; A44C 7/00
USPC 63/12; 422/292; 422/28

(57) ABSTRACT

A backing for a jewelry article includes an outer casing surrounding a chamber storing a disinfectant. Aligned apertures are formed in opposing walls of the backing for allowing passage of a mounting post therethrough. Sealing membranes are retained proximate the apertures to prevent disinfectant from leaking out of the chamber, while permitting the post to be passed therethrough. An absorbent material is housed within the chamber of the outer casing for absorbing the disinfectant disposed within the chamber while permitting the post to pass through such absorbent material. The post is passed into a first aperture, through the chamber, and out of a second aperture for bringing the disinfectant in contact with a majority of the post.

### BACKGROUND OF THE INVENTION

1. Field of the Invention

The present invention relates generally to jewelry and other adornments worn on pierced portions of the human body, and more particularly, to an apparatus and method for disinfecting the posts extending from such items before they are inserted through pierced portions of the body.

2. Description of the Related Art

Body piercing, i.e., the practice of cutting, or otherwise creating, an opening in the human body, for wearing jewelry or other adornments, goes back thousands of years. The remains of ancient mummies show that the practice of piercing the ear lobes has existed for over 5,000 years. Nose piercing was practiced over 3,500 years ago. Today, ornamental jewelry, worn as “piercings”, continues to grow in popularity, and has spread to lips, tongues, eyebrows, and more private body portions.

The posts, or “studs” of jewelry worn as earrings or nose rings is usually made from metal. Surgical stainless steel is often used to reduce the risk of allergic skin reactions. Stainless steel, gold, titanium, and alloys of such metals are also commonly used. While infections often occur shortly after the body is pierced, infections also arise at piercing sites long afterwards. Such infections can arise as a result of frequent removal and replacement, coupled with storage of the removed jewelry items under inadequately-sterile conditions. Often individuals do not bother to wash their hands sufficiently before removing or replacing such jewelry, and bacteria, germs, and/or other foreign bodies are transferred to the studs, and then to the pierced area of the body. Likewise, while it is good practice to sterilize jewelry posts with an antibiotic agent before inserting such jewelry into body piercings, many users simply do not bother, possibly due to the difficulty and inconvenience of carrying sterilizing agents on a regular basis.

U.S. Pat. No. 4,353,370 issued to Evans, and entitled “Medicated Ear Rods And Earring Construction”, describes a medicated ear rod plus a stopper member that is received over the free end of the ear rod. The stopper member includes an external gauze layer and may also be filled with cotton. Alcohol or hydrogen peroxide are poured into the stopper to saturate the external gauze layer and the interior cotton material. While Evans describes the formation of a temporary seal over the ends of the stopper with a thin plastic wrap, Evans further discloses that the temporary seal is broken when the earring is to be used, and that the cleaning agent is continuously supplied to the rod (or as Evans describes it, the “cleaning agent will ooze out of [the] stopper”), and to the pierced ear opening, as the earring is worn.

The apparatus disclosed by Evans is an improvement over unmedicated earrings. The stopper disclosed by Evans is likely very effective for treating already-infected tissue by continuously supplying an antiseptic to such tissue as the earring is worn. However, once the temporary seal described by Evans is broken, the antiseptic agent continues to “ooze” out as the earring is worn. Apart from the possibility of creating an unsightly mess if the antiseptic oozes out too quickly, the supply of antiseptic agent will rapidly be exhausted once the temporary seal is broken. Thus, the user will either need to frequently recharge the antiseptic supply within the Evans stopper, as by re-saturating the gauze layer and interior cotton material, or obtain a fresh stopper after a short period of time. The stopper disclosed by Evans does not lend itself to use over periods of longer duration, at least not without frequent re-charging of the antiseptic.

U.S. Pat. No. 5,639,310 to Giampaolo, Jr., entitled “Method For Manually Disinfecting And Cleaning Post or Pierced Earrings”, disclosed the use of a pad of porous cellular material immersed in disinfecting solution. The pad is normally housed in a re-sealable fluid-tight container. When it is desired to clean and disinfect an earring post, the lid of the container is opened, and the post is inserted into the pad. The post is then removed from the pad, and the lid of the container is re-installed. While undoubtedly effective in disinfecting earring posts, the device disclosed by Giampaolo, Jr. is relatively bulky, and would not be convenient to carry around for use when needed away from the user's home.

Accordingly, it is an object of the present invention to provide a device for disinfecting a post of an article of jewelry to help minimize the risk of infection as such post is inserted into, or removed from, a pierced area of a human body.

It is another object of the present invention to provide such a device for disinfecting a post of an article of jewelry which is relatively inconspicuous.

It is yet another object of the present invention to adequately and evenly apply a disinfectant to such post prior to insertion or removal of the post from the body while avoiding rapid exhaustion of such disinfectant while such article of jewelry is being worn.

It is still another object of the present invention to provide such a device which may be used to retain the post within a pierced opening of the user's body while the article of jewelry is being worn by the user.

A further object of the present invention is to provide such a device which is durable, long-lasting, easy to manufacture and use, small and compact, and relatively inexpensive to construct.

It is also an object of the present invention to provide a method of retaining an article of jewelry on a pierced portion of a human body while disinfecting a post portion of the article of jewelry each time the post is inserted into, or removed from, the pierced portion of the user's body.

These and other objects of the present invention will become more apparent to those skilled in the art as the description of the present invention proceeds.

### SUMMARY OF THE INVENTION

Briefly described, and in accordance with a preferred embodiment thereof, the present invention relates to a device for disinfecting a post of a jewelry article used to adorn a human body. The device includes an outer casing surrounding a chamber. A disinfectant is disposed within the chamber. The chamber includes a first wall having a first aperture formed therein. A first sealing membrane is disposed proximate to the first aperture formed in the first wall of the chamber. The first sealing membrane prevents the disinfectant from leaking out of the chamber through the first aperture, but permits the generally pointed end of the post to repeatedly pass through the first sealing membrane. Thus, the pointed end of the post may be inserted into chamber for disinfecting the post by the disinfectant disposed within the chamber. Preferably, an absorbent material is housed within the chamber of the outer casing for absorbing the disinfectant disposed within the chamber while permitting the post to pass through such absorbent material. This absorbent material could be a sponge, cotton, or similar material.

It is preferred that the device include a first retainer that is secured to the outer casing and which engages the first sealing membrane to keep it proximate to the first wall of the outer casing. The first sealing membrane may be formed of a self-sealing material which permits passage of a pointed post therethrough, and which seals itself against leakage if the post is thereafter removed. Alternatively, the first sealing membrane may include a pre-formed cut therein, whereby the surrounding portions of the first sealing membrane which border upon the pre-formed cut act to seal the pre-formed cut when no post passes therethrough. In either case, the portions of the first sealing membrane surrounding an inserted post seal around the post when a post is present.

Preferably, the aforementioned outer casing of the device includes a second wall opposite the first wall; the second wall has a second aperture formed therein opposite to, and aligned with, the first aperture. In the preferred embodiment, a second sealing membrane is provided proximate to the second aperture, and may be formed, and retained, in the same manner used to form and retain the first sealing membrane. Like the first sealing membrane, the second sealing membrane prevents the disinfectant from leaking out of the chamber through the second aperture, but permits the generally pointed end of the post to repeatedly pass through the second sealing membrane. In this manner, the pointed end of the post may be passed into the first aperture, through the chamber, and out of the second aperture, for allowing the disinfectant to contact a majority of the length of the post.

Another aspect of the present invention is a method of retaining a piece of jewelry or other body adornment on a pierced portion of a human body while disinfecting a post portion of such body adornment. In the preferred form of practicing such method, a backing is provided for the post. The backing includes an outer casing surrounding a chamber; the outer casing includes first and second opposing walls having first and second opposing apertures, respectively, formed therein. The method includes the step of storing a disinfectant within the chamber of the backing. Preferably, the method includes the steps of storing the disinfectant within absorbent material housed within the chamber of the outer casing, and passing the post through the absorbent material. The backing may also be used to retain the body adornment on the pierced portion of the human body.

The method further includes the steps of providing first and second sealing membranes proximate to the first and second apertures, respectively, for preventing the stored disinfectant from leaking out of the chamber through either of the first and second apertures. The post includes a generally-pointed end that can be passed through the first and second sealing membranes. In the preferred embodiment, the method includes the steps of retaining the first and second sealing membranes adjacent to the first and second walls of the outer casing, respectively.

In practicing the inventive method, the post is disinfected by moving the post through the chamber of the backing to distribute disinfectant along the post. Before inserting the post through a pierced portion of the user's body, the disinfected post is removed from the chamber of the backing The pointed end of the post is inserted through the pierced portion of the human body. Then, the pointed end of the post is inserted through the first aperture of the backing, into the chamber, and out through the second aperture of the backing, for applying disinfectant to the post as the post passes within the chamber of the backing

If desired, the aforementioned method may include the steps of pre-forming cuts in the first and second sealing membranes. The portions of sealing membrane which border such pre-formed cuts seal against each other when no post is present, while sealing around the post when the post is passed through each of such sealing membranes.

In an accordance with an alternate embodiment, the present invention is an article of jewelry for being supported upon, and used to adorn, a human body, e.g., an earring, nose ring, diamond stud, or the like. A post or stud has a first end secured to a gem or other adornment and an opposing second end; the second end of the post is generally pointed for passing through a pierced portion of the body. The jewelry article also includes a backing member for releasably engaging the post.

The backing member of the aforementioned jewelry article includes an outer casing that surrounds a chamber and having first and second opposing walls with first and second apertures formed therein and aligned opposite each other. A disinfectant is disposed within the chamber; preferably, a sponge or other absorbent material is also housed within the chamber for absorbing the disinfectant while permitting the post to pass therethrough. First and second sealing membranes are provided proximate to the first and second apertures to prevent the disinfectant from leaking out of the chamber through either of the first and second apertures. In a preferred embodiment, first and second retainers are secured to the outer casing and engage the first and second sealing membranes for retaining them proximate to the first and second walls of the outer casing, respectively.

The first and second sealing membranes permit the second end of the post to pass therethrough. In this manner, the second end of the post may be inserted into the first aperture, through the chamber, and out of the second aperture, for applying disinfectant to the post as the post passes within the chamber of the backing If desired, the first and second sealing membranes may have pre-formed cuts formed therein to facilitate passage of the post therethrough. Portions of the sealing membranes which border upon such pre-formed cuts seal against each other when no post passes therethrough, and seal around the post which a post passes therethrough.

### BRIEF DESCRIPTION OF THE DRAWINGS

FIG. 1 is a perspective view of an article of jewelry and associated backing member in accordance with the present invention.

FIG. 2 is an exploded perspective view of the jewelry article shown in FIG. 1, and showing inner details of the backing member.

FIG. 3 is a perspective, cross-sectional view of the backing member with the outer casing pulled apart, prior to insertion of the post.

FIG. 4 is a side view of the jewelry article shown in FIG. 1 wherein the backing member is shown in sectional view prior to insertion of the post therethrough.

FIG. 5 is a side view of the jewelry article shown in FIG. 1 wherein the backing member is shown in sectional view after insertion of the post therethrough.

FIG. 6 is a detailed view of an alternate sealing membrane having an X-shaped pre-formed cut in the central region thereof.

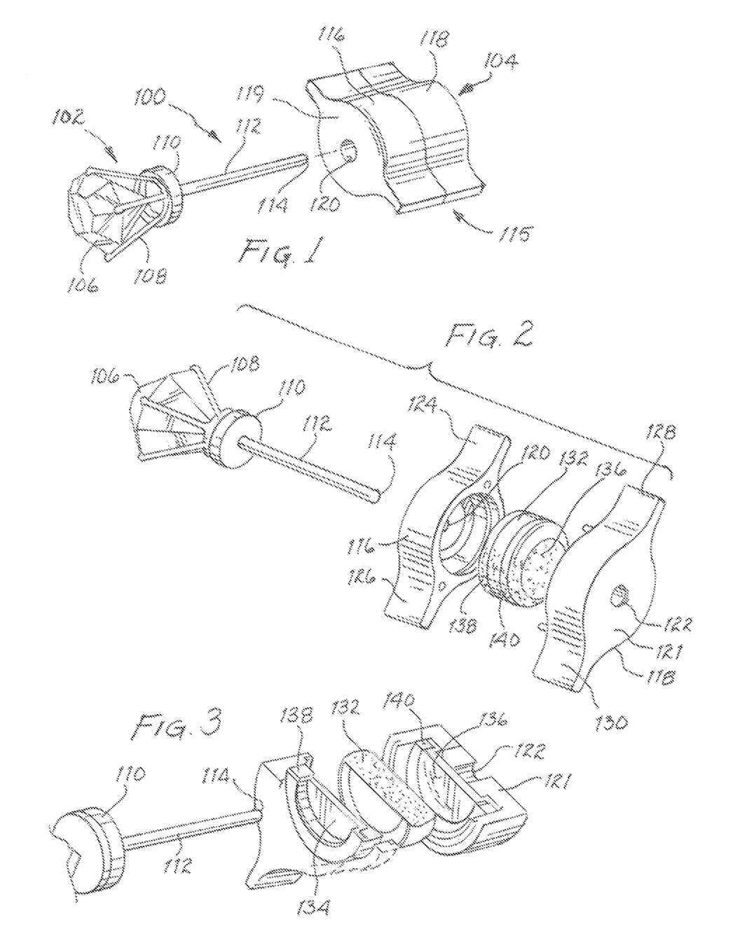

### DETAILED DESCRIPTION OF THE PREFERRED EMBODIMENT

A diamond stud earring for being supported by a pierced portion of the human body, and constructed in accordance with a preferred embodiment of the present invention, is designated generally by reference numeral 100 in FIG. 1. Earring 100 includes stud portion 102 and backing member 104 for releasably engaging stud portion 102. Stud portion 102 includes a gem 106 secured by metal prongs 108 to base 110. Post 112 has a first end secured to base 110 and a second opposing end 114 that is generally rounded and/or pointed for passing the post through a pierced portion of the human body.

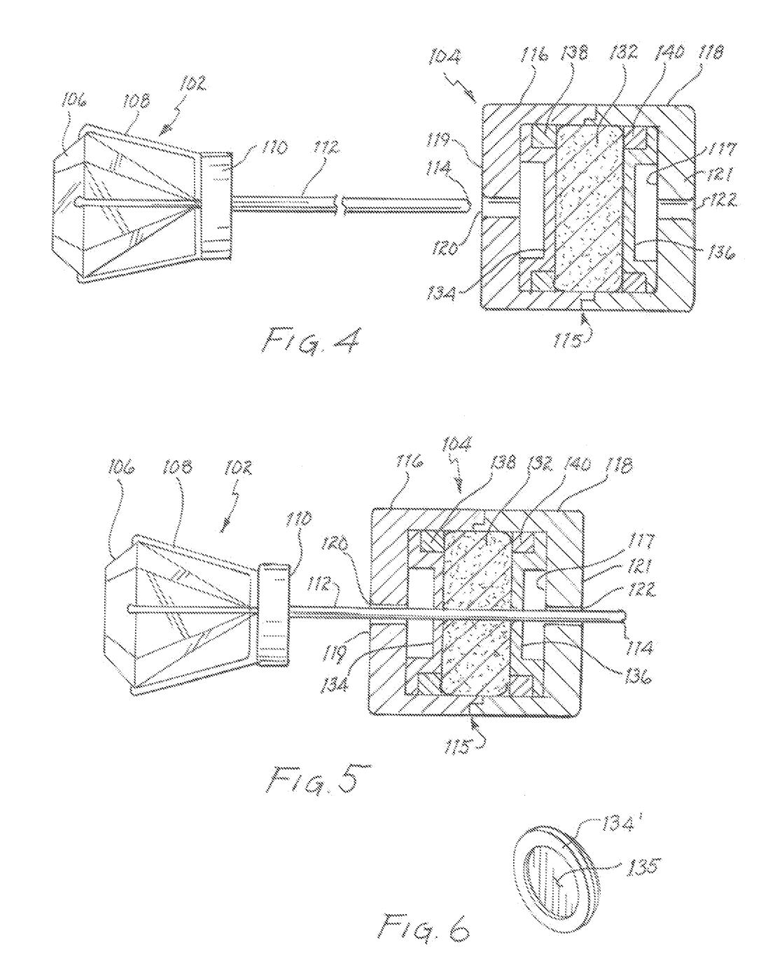

Backing member 104 includes an outer casing 115 preferably formed of two mating halves 116 and 118. As shown best in FIGS. 4 and 5, outer casing 115 surrounds a hollow chamber 117 that can store a disinfectant. As will be explained in greater detail below, backing member 104 may be used to disinfect post 112, either before earring 100 is inserted into the pierced portion of a user's ear, or before earring 100 is removed from the pierced portion of the user's ear. As shown in FIGS. 1, 4 and 5, casing half 116 includes a first wall 119 having an aperture 120 formed therein for receiving post 112. Referring to FIGS. 2, 4 and 5, casing half 118 includes a second wall 121 opposite first wall 119. Second wall 121 has an aperture 122 formed therein opposite aperture 120, and axially aligned therewith, for allowing the generally pointed end 114 of post 112 to exit therethrough. Casing half 116 preferably includes a pair of opposing extension lobes, or wings, 124 and 126. Likewise, casing half 118 preferably includes a pair of opposing extension lobes, or wings, 128 and 130. When casing half 116 is assembled to casing half 118, wings 124 and 128 align with each other, and wings 126 and 130 align with each other, for allowing a user to more easily grip backing member 104. Casing halves 116 and 118 may be formed of injection molded ABS plastic or acrylic material. During assembly, such casing halves 116 and 118 can be joined to each other by a press fit, interlocking tabs and holes, or via sonic welding.

As mentioned above, inner chamber 117 is used to store a supply of disinfectant. Preferably, a sponge 132, or another absorbent material (e.g., cotton) is housed within chamber 117 of outer casing 115. Sponge 132 is saturated with, and absorbs, a disinfectant within chamber 117, while permitting post 112 to be passed therethrough. The absorbed disinfectant may be in liquid or gel form. Preferred disinfectants which may be used include isopropyl rubbing alcohol, iodine, hydrogen peroxide, bacitracin, triclosan or other common topical antibiotics or antibacterial substances.

In order to prevent the disinfectant stored in sponge 132 from leaking out of first aperture 120 in first wall 119, a first sealing membrane 134 is positioned between sponge 132 and first wall 119, generally proximate to first aperture 120. Preferably, first sealing membrane 134 is held in place by a retaining ring 138 which forms a press fit with the inner cylindrical wall of casing half 116 while engaging and urging first sealing membrane 134 against first wall 119. First sealing membrane 134 is preferably made from Fluorosilicone (FSR), Ethylene Propylene Rubber (EPDM), or natural rubber. These materials are resistant to alcohol and are sufficiently compliant to form a reliable seal, even when pierced numerous times. Retaining ring 138 is preferably formed from injection molded ABS plastic or acrylic material; alternatively, retaining ring 138 may also be formed of metal.

If desired, the center section of first sealing membrane 134 may include a thinned region, or even a pre-formed pinhole or cut, to better facilitate passage of pointed end 114 of post 112 therethrough, while still sealing the liquid disinfectant within chamber 117. Such pre-formed cut may be in the form of a single linear slit, in the form of two intersecting slits forming an “X” pattern, or in any other desired form. Referring briefly to FIG. 6, an alternative sealing membrane 134′ is illustrated wherein two intersecting slits form an X-shaped pre-formed cut within the center of sealing membrane 134′. As used herein, the term “pre-formed” cut is intended to include all of the aforementioned pinholes, slits, and cuts. When a pre-formed cut is provided within first sealing membrane 134, the portions of first sealing membrane 134 which border upon such pre-formed cut serve to seal the pre-formed cut when no post passes therethrough. On the other hand, when post 112 is present, as shown in FIG. 5, the portions of first sealing membrane 134 which border upon such pre-formed cut form a sliding sealing around post 112. First sealing membrane 134 not only serves to retain disinfectant inside chamber 117, but also acts as a “wiper” for physically scrubbing contaminants and pathogens on post 112 as it passes through backing 104.

Preferably, a second sealing membrane 136 is provided between sponge 132 and second wall 121, generally proximate to second aperture 122. Second sealing membrane 136 prevents disinfectant from leaking out of chamber 117 through second aperture 122, while permitting generally pointed end 114 of post 112 to pass outwardly through second sealing membrane 136. Second sealing membrane 136 may be constructed of the same material, and in the same fashion described above, for first sealing membrane 134. Similarly, a second retaining ring 140 may be provided for holding second sealing membrane 136 in place within inner chamber 117. Retaining ring 140 forms a press fit with the inner cylindrical wall of casing half 118 while engaging and urging second sealing membrane 136 against second wall 121. Second retaining ring 140 may be constructed of the same material, and in the same fashion described above, for first retaining ring 138.

In use, generally pointed end 114 of post 112 may be passed into first aperture 120 of backing 104, through the first sealing membrane 134, through sponge 132, through second sealing membrane 136, and out of the second aperture 122, for allowing the disinfectant absorbed by sponge 132 to contact those portions of post 112 that pass within chamber 117 of backing 104. Preferably, this operation is performed by the user before inserting jewelry article 100 through the user's pierced ear; backing 104 can be slid along a majority of the length of post 112 to disinfect essentially the entire length of post 112. Backing 104 can then be temporarily removed from post 112 for allowing the user to insert generally pointed end 114 through a pierced ear. Then backing 104 is reinserted onto post 112 to retain earring 100 upon the user's ear. The compressive forces exerted upon post 112 by first sealing membrane 134, second sealing membrane 136, and sponge 132, are generally sufficient to retain backing 104 on post 112 when jewelry article 100 is being worn by a user, while yielding to a user's intentional efforts to move backing 104 along post 112.

When the user later desires to remove earring 100, backing 104 is pulled off of post 112, once again disinfecting at least the portion of the post that was previously behind the user's ear. Earring 100 can then be withdrawn from the user's pierced ear; in doing so, the portions of the post that were formerly behind the user's ear, and which have just been disinfected by removal of backing 104, are now pulled through the pierced portion of the user's ear.

As noted above, one of the aspects of the present invention is an improved method of retaining an article of jewelry or other body adornment on a pierced portion of a human body while disinfecting a post portion of such item. In practicing a preferred embodiment of such method, one provides backing 104 for post 112 wherein backing 104 includes an outer casing 115 surrounding a chamber 117, and wherein the outer casing 115 includes first and second opposing walls 119/121 having opposing apertures 120/122 formed therein. The preferred method includes storing a disinfectant within chamber 117 of backing 104, and providing first and second sealing membranes 134/136 proximate to the first and second apertures 120/122 for preventing the stored disinfectant from leaking out of chamber 117 through first and second apertures 120/122; nonetheless, first and second membranes 134/136 permit the generally pointed end 114 of the post 112 to pass therethrough.

In carrying out the preferred embodiment of this method, post 112 is disinfected by moving post 112 through chamber 117 of backing 104 to distribute disinfectant along post 112. Thereafter, a user removes the generally pointed end 114 of post 112 from chamber 117 of backing 104, and passes generally pointed end 114 of post 112 through the pierced portion of the human body. Then, the user inserts generally pointed end 114 of post 112 into first aperture 120 of backing 104, through chamber 117, and out second aperture 122 of backing 104, for applying disinfectant to post 112 as it passes within chamber 117 of backing 104. Thus, backing 104 serves both to disinfect post 112 and to help retain the article of jewelry on the pierced portion of the human body.

In regard to the step of storing disinfectant within chamber 117, the method preferably includes the steps of providing absorbent material 132 within chamber 117, storing the disinfectant within absorbent material 132, and passing post 112 through absorbent material 132. In its preferred form, the aforementioned method also includes the steps of retaining the first and second sealing membranes 134/136 adjacent to the first and second walls 119/121 of outer casing 115, respectively. According to one preferred embodiment, pre-formed cuts 135 are formed in the sealing membranes 134′, and the portions of such sealing membranes 134′ that border upon pre-formed cuts 135 are allowed to seal against each other when post 112 is not present, while such bordering portions are allowed to seal around post 122 when it passes through such sealing membranes.

Those skilled in the art will now appreciate that an improved device for disinfecting a post of an article of jewelry has been disclosed to help minimize the risk of infection as such post is inserted into, or removed from, a pierced area of a human body. The disclosed disinfecting backing is relatively compact and inconspicuous, and in many instances, may be used to retain the an article of jewelry on the user's body. In those instances where the disclosed disinfecting backing is too large to be worn, it may still be used to disinfect the post of the jewelry article, both before such jewelry article is installed, and before such jewelry article is removed. The incorporation of sealing membranes ensures that disinfectant will be adequately and evenly applied, while avoiding rapid exhaustion of the disinfectant stored within such backing Thus, the disclosed backing is durable and long-lasting, yet easy to manufacture and use, and relatively inexpensive to construct. In addition, a user is able to obtain the above-described disinfection benefits by using the improved backing in essentially the same manner in which a conventional backing is used, i.e., the user benefits from improved hygiene without even thinking about the disinfection process.

While the present invention has been described with respect to preferred embodiments thereof, such description is for illustrative purposes only, and is not to be construed as limiting the scope of the invention. For example, while the preferred embodiment of the invention has been described and illustrated as being the form of an earring, the benefits of the present invention can be applied to all types of body adornments that include a post for being passed through a body piercing. Similarly, while the jewelry article disclosed herein includes a diamond or other gem stone, the improved backing may be used with many types of jewelry and/or body adornments, even those which lack any gem stones. Various other modifications and changes may be made to the described embodiments by those skilled in the art without departing from the true spirit and scope of the invention as defined by the appended claims.

I claim:

1. A device for disinfecting a post of an article used to adorn a human body, the post including a generally pointed end for passing the post through a pierced portion of the human body, the device comprising in combination:
a) an outer casing surrounding a chamber, the outer casing including a first wall, and having a first aperture formed therein;
b) a disinfectant disposed within the chamber;
c) a first sealing membrane disposed proximate to the first aperture, the first sealing membrane preventing the disinfectant from leaking out of the chamber through the first aperture, while permitting the generally pointed end of the post to pass through the first sealing membrane;
d) whereby the generally pointed end of the post may be inserted through the first aperture for being disinfected by the disinfectant disposed within the chamber.

2. The device recited by claim 1 further including absorbent material housed within the chamber of the outer casing, the absorbent material absorbing the disinfectant disposed within the chamber while permitting the post to pass therethrough.

3. The device recited by claim 2 wherein the absorbent material is a sponge.

4. The device recited by claim 1 further including a first retainer, the first retainer being secured to the outer casing and engaging the first sealing membrane to retain the first sealing membrane proximate to the first wall of the outer casing.

5. The device recited by claim 1 wherein the first sealing membrane has a pre-formed cut therein, the pre-formed cut being bounded by first bordering portions of the first sealing membrane, the first bordering portions sealing the pre-formed cut when no post passes therethrough, the pre-formed cut permitting passage of the post therethrough, and the first bordering portions sealing around the post when a post passes through the pre-formed cut.

6. The device recited by claim 1 wherein the post has a length, and wherein:
a) the outer casing includes a second wall opposite the first wall;
b) the second wall has a second aperture formed therein opposite the first aperture; and
the device further comprising:
c) a second sealing membrane disposed proximate to the second aperture, the second sealing membrane preventing the disinfectant from leaking out of the chamber through the second aperture, while permitting the generally pointed end of the post to pass through the second sealing membrane;
d) whereby the generally pointed end of the post may be passed into the first aperture, through the chamber, and out of the second aperture, for allowing the disinfectant to contact a majority of the length of the post.

7. The device recited by claim 6 including absorbent material housed within the chamber of the outer casing, the absorbent material absorbing the disinfectant disposed within the chamber while permitting the post to pass therethrough.

8. The device recited by claim 7 wherein the absorbent material is a sponge.

9. The device recited by claim 6 further including:
a) a first retainer secured to the outer casing and engaging the first sealing membrane to retain the first sealing membrane proximate to the first wall of the outer casing; and
b) a second retainer secured to the outer casing and engaging the second sealing membrane to retain the second sealing membrane proximate to the second wall of the outer casing.

10. The device recited by claim 6 wherein:
a) the first sealing membrane has a first pre-formed cut therein, the first pre-formed cut being bounded by first bordering portions of the first sealing membrane, the first bordering portions sealing the first pre-formed cut when no post passes therethrough, the first pre-formed cut permitting passage of the post therethrough, and the first bordering portions sealing around the post when a post passes through the first pre-formed cut; and
b) the second sealing membrane has a second pre-formed cut therein, the second pre-formed cut being bounded by second bordering portions of the second sealing membrane, the second bordering portions sealing the second pre-formed cut when no post passes therethrough, the second pre-formed cut permitting passage of the post therethrough, and the second bordering portions sealing around the post when a post passes through the second pre-formed cut.

11. A method of retaining a body adornment on a pierced portion of a human body while disinfecting a post portion of such body adornment, the post including a generally pointed end for passing the post through the pierced portion of the human body, the method including the steps of:
a) providing a backing for the post, the backing including an outer casing surrounding a chamber, the outer casing including first and second opposing walls, the first and second walls having first and second opposing apertures, respectively, formed therein;
b) storing a disinfectant within the chamber of the backing;
c) providing a first sealing membrane proximate to the first aperture for preventing the stored disinfectant from leaking out of the chamber through the first aperture, while permitting the generally pointed end of the post to pass through the first sealing membrane;
d) providing a second sealing membrane proximate to the second aperture for preventing the stored disinfectant from leaking out of the chamber through the second aperture, while permitting the generally pointed end of the post to pass through the second sealing membrane;
e) disinfecting the post by moving the post through the chamber of the backing to distribute disinfectant along the post;
f) removing the generally pointed end of the post from the chamber of the backing following step e);
g) passing the generally pointed end of the post through the pierced portion of the human body following step f); and
h) following step g), inserting the generally pointed end of the post into the first aperture of the backing, through the chamber, and out of the second aperture of the backing;
whereby the backing helps to retain the body adornment on the pierced portion of the human body.

12. The method recited by claim 11 wherein:
i) step b) above includes the step of storing the disinfectant within absorbent material housed within the chamber of the outer casing; and
j) step e) above includes the step of passing the post through the absorbent material.

13. The method recited by claim 11 wherein steps c) and d) above include the steps of retaining the first and second sealing membranes adjacent to the first and second walls of the outer casing, respectively.

14. The method recited by claim 11 wherein step c) above include the steps of:
i) pre-forming a cut in the first sealing membrane, the pre-formed cut being bounded by first bordering portions of the first sealing membrane;
j) allowing the first bordering portions to seal against each other when no post passes through the first sealing membrane; and
k) allowing the first bordering portions to seal around the post when a post passes through the first sealing membrane.

15. The method recited by claim 14 wherein step d) above include the steps of:
l) pre-forming a cut in the second sealing membrane, the pre-formed cut being bounded by second bordering portions of the second sealing membrane;
m) allowing the second bordering portions to seal against each other when no post passes through the second sealing membrane; and
n) allowing the second bordering portions to seal around the post when a post passes through the second sealing membrane.

16. The method recited by claim 11 wherein step e) above includes the steps of:
i) inserting the generally pointed end of the post into the backing through one of the first and second apertures, into the chamber, and through the other of the first and second apertures for applying disinfectant to the post as the post passes within the chamber of the backing

17. An article used to adorn a human body and comprising in combination:
a) an adornment for being supported upon the human body;
b) a post having a first end secured to the adornment, and having an opposing second end, the second end of the post being generally pointed for passing the post through a pierced portion of the human body;
c) a backing member for releasably engaging the post, the backing member including:
i) an outer casing surrounding a chamber, the outer casing including first and second opposing walls, the first wall having a first aperture formed therein, and the second wall having a second aperture formed therein opposite the first aperture;
ii) a disinfectant disposed within the chamber;
iii) a first sealing membrane disposed proximate to the first aperture, the first sealing membrane preventing the disinfectant from leaking out of the chamber through the first aperture, while permitting the second end of the post to pass through the first sealing membrane; and
iv) a second sealing membrane disposed proximate to the second aperture, the second sealing membrane preventing the disinfectant from leaking out of the chamber through the second aperture, while permitting the second end of the post to pass through the second sealing membrane;
d) whereby the second end of the post may be inserted into the first aperture, through the chamber, and out of the second aperture, for applying disinfectant to the post as the post passes within the chamber.

18. The article recited by claim 17 further including absorbent material housed within the chamber of the outer casing, the absorbent material absorbing the disinfectant disposed within the chamber while permitting the post to pass therethrough.

19. The article recited by claim 18 wherein the absorbent material is a sponge.

20. The article recited by claim 17 further including:
a) a first retainer secured to the outer casing and engaging the first sealing membrane to retain the first sealing membrane proximate to the first wall of the outer casing; and
b) a second retainer secured to the outer casing and engaging the second sealing membrane to retain the second sealing membrane proximate to the second wall of the outer casing.

21. The article recited by claim 17 wherein:
a) the first sealing membrane has a first pre-formed cut therein, the first pre-formed cut being bounded by first bordering portions of the first sealing membrane, the first bordering portions sealing the first pre-formed cut when no post passes therethrough, the first pre-formed cut permitting passage of the post therethrough, and the first bordering portions sealing around the post when a post passes through the first pre-formed cut; and
b) the second sealing membrane has a second pre-formed cut therein, the second pre-formed cut being bounded by second bordering portions of the second sealing membrane, the second bordering portions sealing the second pre-formed cut when no post passes therethrough, the second pre-formed cut permitting passage of the post therethrough, and the second bordering portions sealing around the post when a post passes through the second pre-formed cut.
